(19) United States
(12) Patent Application Publication
(10) Pub. No.: US 2004/0039445 A1
(43) Pub. Date: Feb. 26, 2004

(54) Orbital implant and method

(76) Inventor: Maine Grip, Vollen (NO)

Correspondence Address: CHRISTIAN D. ABEL, ONSAGERS AS, POSTBOKS 6963 ST. OLAVS PLASS, NORWAY, N-0130, NO

(21) Appl. No.: 10/381,188

(22) PCT Filed: Jun. 21, 2001
(86) PCT No.: PCT/NO01/00262

(30) Foreign Application Priority Data
Sep. 29, 2000 (US) 60236343

(51) Int. Cl. A61F 2/14

(52) U.S. Cl. 623/4.1; 623/6.64

(57) ABSTRACT

A supplementary implant for ameliorating enophtalmos comprises a plate (10) having a plurality of attachment holes (14), with a posterior, generally spherical projection (12) adapted to replace the volume of soft tissue loss resulting from eye enucleation, and a method for attaching said implant to the floor of the orbit by screws or stainless steel ligatures posterior to an orbital implant.

### TECHNICAL FIELD

This invention relates to an orbital implant and method, and more particularly to a supplemental orbital implant shaped to reduce the soft tissue deformity of the eye socket attributed to enophthalmos.

### BACKGROUND ART

When an eye is removed due to trauma, tumor or other disease states, the eyeball is usually replaced with a spherical implant made of either silicone or hydroxyapatite, such as that from U.S. Pat. No. 4,976,731. After surgical healing, an artificial eye prosthesis is prepared in either glass or plastic material. In the case of hydroxyapatite implants, the eye muscles can be sutured directly to the implant and thus confer movement to the implant. By a special peg attachment, this movement can be transferred to the artificial eye prosthesis and allows it to move in conjunction with the companion eye. A natural, lifelike movement of the artificial eye is thus possible and offers many benefits to patients wearing an artificial eye. Also, the peg supports the weight of the artificial eye and reduces pressure and strain on the lower eyelid.

Despite maximal efforts to create a well adapted and well functioning artificial eye connected to a tissue-integrated hydroxyapatite spherical implant, the majority of patients over time take on an unnatural shrunken appearance of the eyelid and orbit. This condition is due to atrophy or sinking back of the soft tissues of the orbit and is referred to as enophthalmos. Clinically, enophthalmos is specially manifested by a finding of deep superior sulcus in the form of a hollowing out of the upper eyelid and retarded position of the artificial eye prosthesis. Increasing the size of the ocular prosthesis such as shown in U.S. Pat. No. 5,466,258 can to a certain extent mitigate this disfiguring. However, the increased weight of a larger prosthesis adds to the strain on the lower eyelid and is seldom cosmetically successful.

### SUMMARY OF THE INVENTION

The present invention is intended to overcome the above-described problems. The implant according to the invention is designed to be a supplement to orbital implants currently in use, by compensating for the loss of soft tissue bulk in the eye socket. The implant is made of either polyethylene, polymethylmetacrylate, Teflon® or other suitable material. The implant comprises an approximately 1.5 cm×3-4 cm plate, approximately 2 mm thick, adapted to the shape of the orbital floor. The plate has a plurality of attachment holes and a posterior, generally spherical projection, said projection providing added volume replacement for the lost soft tissue volume resulting from eye enucleation. The projection further exerts a forward pressure on the existing soft tissue, spherical ocular implant, eye prosthesis and eyelids, thus helping to eliminate the unnatural sunken appearance. The implant may be produced in a plurality of standard sizes adapted to varying degrees of volume replacement, and may be shaped by the surgeon according to the situation. In an alternate aspect of the invention, the implant could be utilized where the patient's natural eye, for one reason or another, lies deeper in the eye socket than normal.

The implant according to the invention may be utilized as part of an initial procedure, or as a corrective measure for an earlier, now unsatisfactory procedure. The method according to the invention comprises determining and selecting the appropriate standard-sized implant, modifying the size or shape of the projection as necessary, adjusting the length of the implant by cutting the anterior part of the plate, and the subperiosteal (via a subciliary incision in the lower eyelid) implantation of the implant in the floor of the orbit. The implant is securely fixed to the orbital floor by two stainless-steel ligatures that are anchored to drill holes in the inferior orbital rim, or by screws. Due to the bony fixation and the enwrapment in a subperiosteal pocket, the implant is totally immobilized and is not the subject of post-operative drifting or dislocation. The implant according to the invention is ideally combined with a spherical hydroxyapatite ocular implant, which during the last decade has gained increased popularity among ophthalmologists around the world. Together, these two implants offer significant esthetic improvement of the disfiguring enophthalmos condition.

### BRIEF DESCRIPTION OF THE DRAWINGS

FIG. 1 is a perspective view of the orbital implant of the invention;

FIG. 2 is a side elevational view of the orbital implant of the invention;

FIG. 3 a bottom plan view of the orbital implant of the invention;

FIG. 4 is a perspective view of the orbital implant of the invention;

FIG. 5 is a sectional view showing the implant in place in an eye socket occupied by an ocular implant and prosthesis.

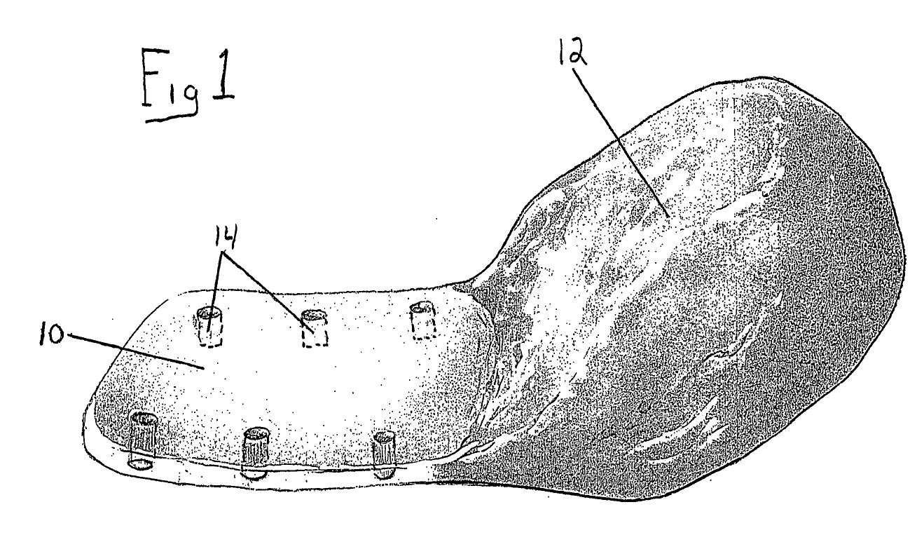

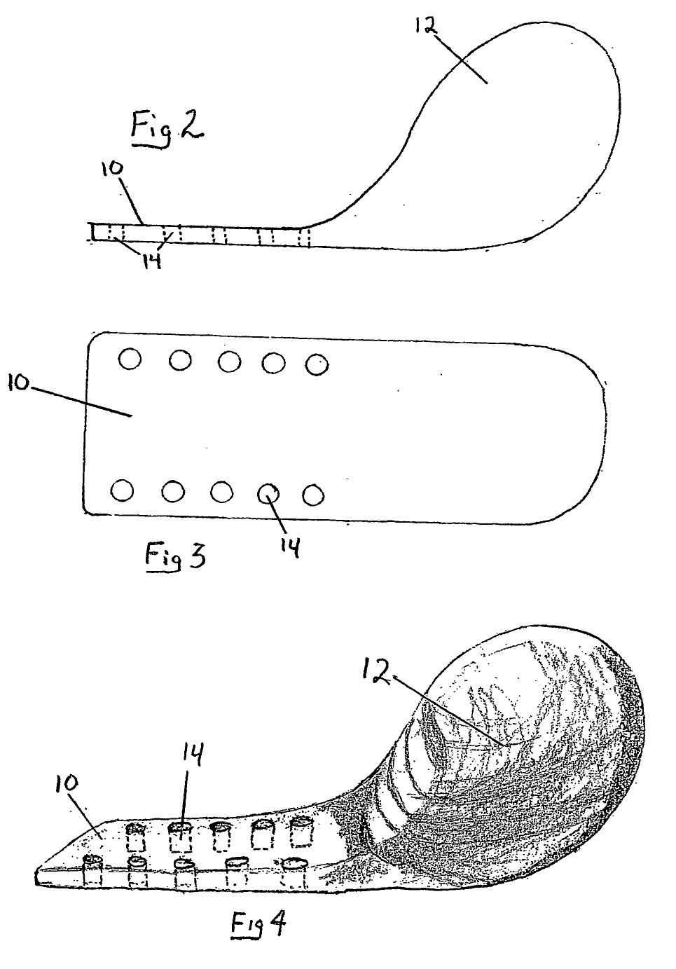

### DETAILED DESCRIPTION OF THE INVENTION

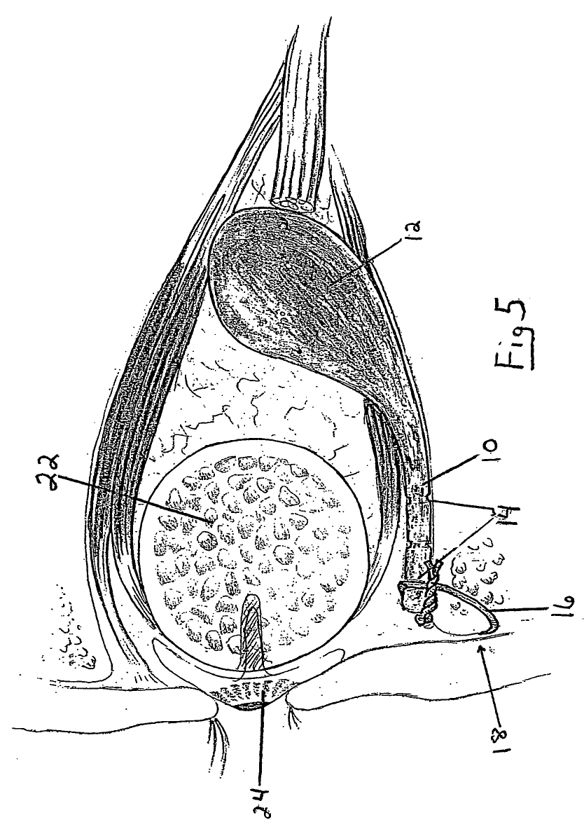

As shown in the figures, the implant according to the invention comprises a plate 10 with a posterior, generally spherical projection 12. Plate 10 further comprises two rows of a plurality of attachment holes 14. As can be appreciated from FIG. 3, the length of plate 10 may be adjusted by cutting away a portion of the anterior end of the plate. FIG. 5 illustrates the implant according to the invention affixed to the orbital floor by a stainless-steel ligature 16. As can be discerned from FIG. 5, ligature 16 passes through a drill hole 18 in the inferior orbital rim. It is to be understood, however, that the implant could alternatively be attached by screws. The implant is positioned behind an ocular implant 22 to which is connected an eye prosthesis 24. As can be appreciated, spherical projection 12 serves to replace lost soft-tissue volume and press ocular implant 22 forward, thus mitigating the enophthalmos condition. According to the preferred embodiment, projection 12 is generally spherical, however any appropriate, space-filling shape could be employed.

1. A supplemental orbital implant for ameliorating enophthalmos comprising a plate (10) having an anterior and a posterior end, a space-filling projection (12) extending from the posterior end and a plurality of attachment holes (14) arranged in said plate (10).

2. An orbital implant according to claim 1, wherein said space-filling projection (12) is generally spherical.

3. An orbital implant according to claim 2, wherein projection (12) extends above the horizontal plane defined by plate (10).

4. An orbital implant according to claim 3, wherein holes (14) are arranged in pairs, along two parallel rows, such that the length of plate (10) can be adjusted by cutting away a desired portion of the anterior end leaving at least one pair of attachment holes (14) in plate (10).

5. An orbital implant according to claim 4, wherein the dimensions of plate (10) are adapted to the dimensions of the orbital floor and the dimensions of projection (12) are predetermined to approximate the lost soft-tissue volume resulting from eye enucleation.

6. An orbital implant according to claim 5, wherein said implant is constructed of either polyethylene, polymethylmetacrylate, Teflon® or other suitable material.

7. A method for ameliorating enophthalmos comprising the steps of:
a) examining a patient to determine the amount of soft tissue loss resulting from eye enucleation;
b) providing an implant of the type described in either of claims 1-6;
c) modifying the dimensions of projection (12) as necessary;
d) adjusting the length of plate (10) as necessary;
e) implanting the implant in the eye socket of the patient posterior to an orbital implant.

8. A method for ameliorating enophthalmos according to claim 7, wherein said implant is attached to the floor of the orbit by screws.

9. A method for ameliorating enophthalmos according to claim 7, further comprising the steps creating drill holes in the inferior orbital rim and attaching said implant to said drill holes with stainless steel ligatures or the like.

10. A method for ameliorating enophthalmos according to either claim 8 or claim 9, wherein said implant is inserted via a subciliary incision in the lower eye lid.
